(19) United States
(12) Patent Application Publication
(10) Pub. No.: US 2025/0215392 A1
(43) Pub. Date: Jul. 3, 2025

(54) SYSTEM AND PROCESSES FOR CULTURING NON-HUMAN-ANIMAL CELLS UNDER VARIABLE GRAVITY CONDITIONS

(71) Applicant: ALEPH FARMS LTD., Rehovot (IL)

(72) Inventors: Neta Lavon, Ness Ziona (IL); Zvi Tamari, Givat Shmuel (IL)

(21) Appl. No.: 18/850,044

(22) PCT Filed: Mar. 23, 2023
(86) PCT No.: PCT/IL2023/050306

§ 371 Date: Sep. 23, 2024

Related U.S. Application Data
(60) Provisional application No. 63/323,093, filed on Mar. 24, 2022.

(51) Int. Cl. C12N 5/073; A23L 13/00; C12M 1/00; C12M 1/34; C12M 1/36

(52) U.S. Cl. CPC C12N 5/0603; A23L 13/00; C12M 23/34; C12M 41/30; C12M 41/48; C12N 2506/45; C12N 2525/00; C12N 2533/70; C12N 2537/10

(57) ABSTRACT

The present invention relates to the expanding field of research relating to long-term living of humans in space environment, particularly to systems and processes for the production of high-quality, high-protein nutritional products, particularly for the production of cultured meat products in space.

### FIELD OF THE INVENTION

The present invention relates to the expanding field of research relating to long-term living of humans in space environment, particularly to systems and processes for the production of high-quality, high-protein nutritional products, particularly for the production of cultured meat products in space.

### BACKGROUND OF THE INVENTION

Prolonged stay in space, including long-duration manned missions and human space outposts, is challenged by the ability to provide quality nutrition to humans while in space. To date, all crewed space missions have been short-term and in a relatively low earth orbit, and rely on food replenishment from earth. In the future, long-term missions and permanent planetary bases such as those on the moon and Mars, are to be common, and in these situations, it will not be possible to supply food from earth.

High-density food such as beef steaks is traditionally obtained from mammals, in a process which needs to transform large quantities of feeders and water into meat (over 25 kg of feed and 10,000 liters of water per kg of beef). This is a highly inefficient process in terms of Feed Conversion Ratio (FCR), whereby a lot of energy is used for the indirect transformation of inputs into edible tissue. Furthermore, only about 250 kg may be used as consumable food out of an average cow weight of 600 kg.

In contrast, cultivated meat is produced using optimal medium concentration (water, sugars, amino acids, fatty acids etc.) at the cellular level. The Applicant of the present invention and others have developed compositions, methods, and systems for producing cultured meat under terrestrial (earth gravity) conditions.

One major challenge in culturing cells in the outer space is the lack of effective gravity, e.g., while orbiting Earth, or reduced gravity such as that present on the Moon or Mars. Cellular traits and processes have evolved for millions of years in the presence of gravitational force and are profoundly affected by its absence. Indeed, many studies have shown that cellular characteristics such as morphology, cell cycle, biosynthetic processes and cell signaling are significantly altered in microgravity environment.

Attempts for performing particular steps in processes for cell proliferation and differentiation under partial and/or microgravity conditions, for various uses, have been also made. For example, International (PCT) Application Publication No. WO 03/087292 discloses systems, modules, bioreactor and methods for the automated culture, proliferation, differentiation, production, and maintenance of tissue engineered products. The tissue engineering system and components thereof are operable under conditions of microgravity and/or zero gravity where such system and components are used for space research.

Pluripotent stem cells (PSCs) are cells that have the capacity to self-renew by dividing while keeping the capability to differentiate to every cell type in the body. Both embryonic stem cells (ESCs) and induced pluripotent stem cells (iPSCs) are pluripotent stem cells. ESCs are typically derived from the cell population in the inner cell mass of pre-implantation blastocysts. Induced pluripotent stem cells (iPSCs) are a type of PSCs. which are generated from somatic differentiated cells reprogrammed to recover their pluripotency characteristics. PSCs are widely used as a starting culture for further differentiation and production of engineered tissues, both for medical purposes and as a food (cultured meat).

U.S. Pat. No. 7,588,938 discloses a method of growing tissue comprising the steps of providing primary mammalian stem cells and progenitor cells, placing the stem cells and the progenitor cells in an extracellular matrix, and maintaining the matrix in a culture medium and a microgravity environment, the method resulting in the formation of a tissue.

U.S. Pat. No. 8,993,231 discloses methods for rapidly expanding a stem cell population with or without culture supplements in simulated microgravity conditions. The invention also relates to methods for increasing the sensitivity of cancer stem cells to chemotherapeutic agents by culturing the cancer stem cells under microgravity conditions and in the presence of omega-3 fatty acids, and for testing the sensitivity of cancer cells and cancer stem cells to chemotherapeutic agents by culturing the cancer cells and cancer stem cells under microgravity conditions. The methods of the invention can also be used to produce tissue for use in transplantation by culturing stem cells or cancer stem cells under microgravity conditions.

U.S. Pat. No. 10,696,951 discloses a method for culturing pluripotent stem cells by culturing an isolated pluripotent stem cells in a pseudo-microgravity environment to proliferate the pluripotent stem cells while maintaining the pluripotent stem cells in an undifferentiated state, thereby forming and growing spheroids of the pluripotent stem cells. The invention further discloses a method for inducing differentiation of pluripotent stem cells by using the method.

A challenge in the production of cultured meat, both under earth and space conditions, is the need to grow large quantities of cells in bioreactors or cell-growth chambers under three-dimensional (3D) conditions. Use of hydrogels as a 3D growth matrix has been proposed, particularly in research relating to clinical tissue engineering. Goetzke et al. reported that differentiation of induced pluripotent stem cells towards mesenchymal stromal cells is hampered by culture in 3D hydrogels (Goetzke R et al. 2019. Scientific Reports 9:15578; doi.org/10.1038/s41598-019-51911-5).

Kapr et al. describe human induced pluripotent stem cell-derived neural progenitor cells that produce distinct neural 3D-in vitro models depending on hydrogel blend properties, and showed a blend of alginate/gellan gum/laminin to be highly suitable for producing 3D neuronal network (Kapr J et al. 2021. Adv. Healthcare Mater. 10:2100131).

There is an emerging need, and it would be highly beneficial to have, systems and processes for producing cultured meat products under variable gravity conditions, including partial, micro- and zero-gravity as a high nutritional source for human living in space environment for long terms.

### SUMMARY OF THE INVENTION

The present invention answers the above-described needs, providing processes and systems that can be used for the production of cells, tissues, and cultured meat products comprising same under partial, micro- or zero-gravity conditions, particularly when such conditions are present out of Erath, in orbiting vehicles or in space settlements, for example on Mars or Moon.

Using the system and processes of the invention, further provided are methods for studying the characteristics of cells proliferated and differentiated under partial or zero-gravity conditions, and optimizing the cell culturing processes under these gravity conditions, particularly cell culturing for the production of cultured meat. The present invention further provides cultured meat products based on the processes described herein.

The present invention is based in part on the unexpected finding that bovine derived pluripotent stem cells are capable of proliferating and thereafter differentiating into mesoderm committed cells while embedded within a semi-solid or solid polysaccharide hydrogel, particularly sodium-alginate hydrogel. The present invention further discloses that alginate concentration of up to about 0.8% w/v is preferable for maintaining the cell capability to proliferate. Furthermore, proliferation and differentiation of the bovine-derived pluripotent stem cells was obtained when the cell-containing semi-solid or solid alginate hydrogel was placed under gravity conditions (in an earth-located facility) as well as under micro- or zero-gravity (in a spacecraft).

According to certain aspects, the present invention provides a process for producing mesoderm-committed non-human-animal cells (MCNHACs) under variable gravity conditions, the process comprising the steps of:
a. seeding a plurality of non-human-animal pluripotent stem cells (NHAPSCs) in a suspension comprising cell-culture medium comprising at least one type of polysaccharide;
b. inducing conditions enabling the transition of the suspension to a solid or semisolid state, thereby forming a solid or semisolid hydrogel comprising the NHAPSCs;
c. maintaining the solid or semisolid hydrogel comprising the NHAPSCs under proliferation conditions for a duration enabling proliferation of said NHAPSCs; and
d. replacing the proliferation conditions to differentiation conditions and maintaining the differentiation conditions for a duration enabling the differentiation of said NHAPSCs to MCNHACs;
thereby producing a solid or semisolid hydrogel comprising a plurality of MCNHACs.

According to certain embodiments, the proliferation conditions comprise incubation the NHAPSCs in a cell culture medium comprising components that promote proliferation of said NHAPSCs.

According to certain embodiments, the duration enabling the proliferation of the NHAPSCs is at least 4 days.

According to certain exemplary embodiments, the duration enabling the proliferation of the NHAPSCs is from about 4 days to about 15 days.

According to certain embodiments, the differentiation conditions comprise incubation of the NHAPSCs in a cell culture medium comprising components that promote differentiation of said NHAPSCs to MCNHACs.

According to certain embodiments, the duration enabling the differentiation of the NHAPSCs to MCNHACs is at least 3 days.

According to certain exemplary embodiments, the duration enabling the differentiation of the NHAPSCs to MCNHACs is from about 3 days to about 15 days.

According to certain embodiments, at least part of steps (a) to (d) are performed within a system comprising at least one compartment. According to certain exemplary embodiments, the entire process comprising steps (a) to (d) is performed within the system.

According to certain embodiment, the system is configured to perform at least one of steps (a)-(d) automatically. According to certain exemplary embodiments the system is configured to perform the entire process, comprising steps (a)-(d), automatically.

According to certain embodiments, the variable gravity conditions are selected from the group consisting of earth gravity, partial gravity, micro-gravity and zero gravity conditions. Each possibility represents a separate embodiment of the present invention.

According to some embodiments, the entire process is performed under earth gravity conditions.

According to certain alternative embodiments, the entire process is performed under partial gravity conditions.

According to yet further certain alternative embodiments, the entire process is performed under micro- or zero-gravity conditions.

According to certain embodiments, steps (a)-(b) are performed under earth gravity conditions, and steps (c)-(d) are performed under partial, micro- or zero-gravity conditions.

According to certain embodiments, the at least one type of polysaccharide is selected from the group consisting of alginate, starch, bean, gum, gellan-gum, hyaluronic acid, cellulose, chitin, chitosan, xanthan gum, agar, agarose, pectin, dextran, carrageenan, salts thereof, modifications and/or variations thereof, and combinations thereof. Each possibility represents a separate embodiment of the present invention.

According to certain exemplary embodiments, the at least one polysaccharide is selected from the group consisting of alginate and RGD-modified alginate. Each possibility represents a separate embodiment of the present invention.

According to further exemplary embodiments, the at least one polysaccharide is alginate.

According to yet additional exemplary embodiments, the at least one polysaccharide is RGD-modified alginate.

According to certain embodiments, the concentration of the at least one polysaccharide is from about 0.3 to about 0.8% w/v based on the volume of the suspension.

According to certain exemplary embodiments, the at least one polysaccharide is alginate, present at a concentration of about 0.4% w/v based on the volume of the suspension.

According to certain embodiments, the conditions enabling the transition of the suspension to a solid or semisolid state comprise exposing said suspension to at least one crosslinking mechanism. According to some embodiments, the crosslinking mechanism is selected from the group consisting of chemical crosslinking, thermal crosslinking, photopolymerization, enzymatic polymerization, and combinations thereof. According to certain exemplary embodiments, the chemical crosslinking comprises adding to the suspension at least one divalent or trivalent ion selected from the group consisting of calcium (Ca⁺²), Barium (Ba⁺²), Copper (Cu⁺²), magnesium (Mg⁺²), iron (Fe⁺² and/or Fe⁺³), Aluminum (Al⁺³), and salts thereof. According to further exemplary embodiments, the divalent ion is calcium (Ca⁺²) or a salt thereof.

According to certain embodiments, the cell-culture medium is a serum free medium. According to certain embodiments, the cell-culture medium is animal-derived component-free medium.

According to some embodiments, the cell culture medium further comprises at least one supplement selected from the group consisting of a colorant, a vitamin, a flavoring agent, folate, zinc and/or a salt thereof, selenium and/or a salt thereof, Coenzyme Q10, at least one fatty acid, yeast extract, bacterial extract, and any combination thereof. Each possibility represents a separate embodiment of the present invention. According to certain embodiments, the cell culture medium forms the basis of the proliferation medium and the differentiation medium.

According to certain embodiments, the system is configured to automatically inject proliferation medium and/or differentiation medium through the solid or semisolid hydrogel comprising the non-human-animal cells in a continuous manner and/or at pre-determined intervals. According to certain embodiments, the system is configured to automatically inject proliferation medium and/or differentiation medium through the solid or semisolid hydrogel comprising the non-human-animal cells at pre-determined volumes. According to certain embodiments, the system is configured to automatically inject proliferation medium and/or differentiation medium through the solid or semisolid hydrogel comprising the non-human-animal cells at pre-determined flow rate.

According to certain embodiments, the system is configured to automatically inject proliferation and/or differentiation medium through the solid or semisolid hydrogel comprising the non-human-animal cells in a continuous manner.

According to certain embodiments, the system is configured to automatically inject proliferation medium through the solid or semisolid hydrogel comprising the NHAPSCs once every about 12 hours with pre-determined injection volume and flow rate.

According to certain embodiments, the system is configured to automatically inject differentiation medium through the solid or semisolid member comprising the NHAPSCs and/or MCNHACs once every about 24 hours with pre-determined injection volume and flow rate. According to certain embodiments, wherein the process or parts thereof is performed within the system, said system comprises at least one compartment comprising the solid or semisolid hydrogel comprising a plurality of non-human-animal cells and at least one additional compartment. The non-human-animal cells can be NHAPCSc, MCNHACs and a combination thereof.

According to certain embodiments, the system is further configured to maintain a pre-set temperature in its at least one compartment. According to some embodiments, the system is configured to maintain a pre-set temperature in the compartment comprising the non-human-animal cells containing solid or semi solid hydrogel, wherein the pre-set temperature is selected from the group consisting of a temperature in the range selected from the group consisting of from about 4° C. to about 15° C., from about 35° C. to about 39° C. According to certain embodiments, the pre-set temperature is selected from the group consisting of about 4° C., about 37° C. and 38.5° C. Each possibility represents a separate embodiment of the present invention.

The non-human-animal cells can be NHAPCSc and/or MCNHACs.

According to certain embodiments, the system is further configured to automatically obtain microscopic images of the solid or semisolid hydrogel and/or the cells therein.

According to certain embodiments the process further comprises step (e) of replacing the differentiation conditions to maturation conditions enabling further differentiation of said MCNHACs to at least one of type of cells selected from the group consisting of muscle cells, adipocytes, fibroblasts, endothelial cells, collagen producing cells and any combination thereof. Each possibility represents a separate embodiment of the present invention.

According to certain exemplary embodiments, the process is used for producing cultured meat products. According to these embodiments, components of the culture medium are preferably edible. According to certain exemplary embodiments, the at least one polysaccharide is edible.

According to certain embodiments, the cultured meat produced by the process of the present invention comprises an edible cross-linked polysaccharide and non-human-animal cells selected from the group consisting of mesenchymal-committed non-human-animal cells (MSNHACs), muscle cells, adipocytes, fibroblasts, endothelial cells, collagen producing cells and any combination thereof. Each possibility represents a separate embodiment of the present invention.

According to certain embodiments, the cultured meat produced by the process of the present invention comprises an edible cross-linked polysaccharide and MCNHACs. According to certain additional or alternative embodiments, the cultured meat produced by the process of the present invention comprises an edible crosslinked polysaccharide and at least one type of cells selected from the group consisting of muscle cells, adipocytes, fibroblasts, endothelial cells, collagen producing cells and any combination thereof.

According to some embodiments, the process further comprises supplementing the resulted cultured meat with at least one supplement selected from the group consisting of a colorant, a vitamin, a flavoring agent, folate, zinc and/or a salt thereof, selenium and/or a salt thereof, Coenzyme Q10, at least one fatty acid, yeast extract, bacterial extract, amino acids, non-animal protein and any combination thereof. Each possibility represents a separate embodiment of the present invention.

According to certain embodiments, the process in its entirety is performed within a closed system.

According to certain exemplary embodiments, the micro- or zero-gravity conditions are outer space conditions.

According to additional aspects, the present invention provides a cultured meat product comprising at least one cross-linked edible polysaccharide and at least one type of non-human animal cells, produced by the process of the present invention.

According to certain embodiments, the non-human-animal cells are selected from the group consisting of mesenchymal-committed cells, muscle cells, adipocytes, fibroblasts, endothelial cells, collagen producing cells and any combination thereof, and any combination thereof. Each possibility represents a separate embodiment of the present invention.

According to certain embodiments, the cultured meat product produced by the process of the present invention further comprises at least one supplement selected from the group consisting of a colorant, a vitamin, a flavoring agent, folate, zinc and/or a salt thereof, selenium and/or a salt thereof, Coenzyme Q10, at least one fatty acid, yeast extract, bacterial extract, non-animal protein and any combination thereof. Each possibility represents a separate embodiment of the present invention.

According to further aspects, the present invention provides a method for obtaining RNA and/or RNA-marker profile of cells differentiating under variable gravity condition, comprising the steps of:

in a first system and a second system for cell and/or tissue culturing comprising at least one compartment:
a. seeding a plurality of non-human-animal pluripotent stem cells (NHAPSCs) in a suspension comprising cell-culture growth medium comprising at least one type of polysaccharide;
b. inducing conditions enabling the transition of the suspension to a solid or semisolid state, to thereby forming a solid or semisolid hydrogel comprising the NHAPSCs;
c. maintaining said solid or semisolid hydrogel comprising the NHAPSCs under proliferation conditions for a duration enabling proliferation of said NHAPSCs;
d. replacing the proliferation conditions to differentiation conditions and maintaining the differentiation conditions for a duration enabling the differentiation of said NHAPSCs to mesoderm committed non-human animal cells (MCNHACs) and optionally to cells differentiated therefrom to obtain solid or semisolid hydrogel comprising differentiated non-human animal cells; and
e. stabilizing cellular RNA in situ within said semisolid or solid under unfrozen conditions;
wherein in the first system at least part of steps (a)-(e) are performed under gravity conditions selected from the group consisting of partial, micro- or zero-gravity conditions and in the second system the entire steps (a)-(e) are performed under earth gravity conditions.

According to certain embodiments, the duration enabling the proliferation of the NHAPSCs is at least 4 days.

According to certain embodiments, the duration enabling the differentiation of the NHAPSCs to MCNHACs is at least 3 days.

According to certain embodiments, the method further comprises sampling RNA from the cells comprised within the solid or semisolid hydrogel of each of the first and the second systems. According to certain embodiments, the sampled RNA is subjected to a quantification analysis. According to certain embodiments, the sampled RNA is subjected to at least one of RNA sequencing and RNA markers analysis and any combination thereof. According to certain embodiments, RNA sequencing and/or RNA marker analysis provides RNA and/or RNA-marker profile characteristic to cells comprised in each of the first and the second systems. According to certain embodiments, the method further comprises comparing the RNA quantity, and/or RNA sequence and/or RNA-marker profile obtained from the first system to those obtained from the second system.

According to certain exemplary embodiments, the partial, micro- or zero-gravity conditions are outer-space conditions.

According to yet further aspects, the present invention provides a system for producing cultured meat product under partial, micro- or zero-gravity conditions, the system comprising:
a. at least one storage compartment configured to store frozen non-human-animal pluripotent stem cells (NHAPSCs);
b. at least one growth compartment configured, under sterile or semi-sterile condition to (i) process NHAPSCs seeding, proliferation, and optionally differentiation, as to obtain mesenchymal-committed non-human-animal cells (MCNHACs) and optionally differentiated non-human-animal cell and/or a tissue comprising same forming a meat portion; and (ii) maintain the differentiated non-human-animal cells and/or tissue comprising same;
c. at least one cooking compartment with means for cooking the meat portion;
d. means for transforming the NHAPSCs from the at least one storage compartment to the at least one growth compartment;
f. means for transforming the meat portion from the at least one growth compartment to the at least one cooking compartment;
g. means for supplying at least one edible liquid substance into the at least one growth compartment; optionally,
h. means for supplying at least one edible solid substance into the at least one growth compartment;
wherein the system is a closed system and wherein the compartments and means are suitable for operation under partial, micro-, or zero-gravity conditions.

According to certain embodiments, the non-human-animal pluripotent stem cells used according to the teachings of the present invention are obtained from an animal selected from the group consisting of ungulate, poultry, aquatic animals, invertebrate and reptiles. Each possibility represents a separate embodiment of the present invention.

According to certain embodiments, the ungulate is selected from the group consisting of a bovine, an ovine, an equine, a pig, a giraffe, a camel, a deer, a hippopotamus, and a rhinoceros. Each possibility represents a separate embodiment of the present invention.

According to certain exemplary embodiments the non-human-animal pluripotent stem cells are bovine cells. According to further exemplary embodiments, the bovine is a cow.

According to certain embodiments, the cell differentiated from the non-human-animal pluripotent stem cells are selected from the group consisting of muscle cells, adipocytes, fibroblasts, endothelial cells, collagen producing cells and any combination thereof. Each possibility represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

It is to be understood that any combination of each of the aspects and the embodiments disclosed herein is explicitly encompassed within the disclosure of the present invention.

### BRIEF DESCRIPTION OF THE FIGURES

FIG. 1 shows bovine stem cells after 11 days of proliferation within sodium alginate hydrogel. Aggregates of bovine stem cells at a cell concentration of 106/ml were seeded in a suspension comprising 0.4% w/v alginate. A semi-solid hydrogel was formed after the addition of 100 mM CaCl₂). The hydrogel was supplemented with a proliferation medium for 11 days.

FIG. 2 shows bovine stem cells after 5 days of proliferation followed by 5 days of differentiation. Aggregates of bovine stem cells at a cell concentration of 106/ml were seeded in a suspension comprising 0.4% w/v alginate. A semi-solid hydrogel was formed after the addition of 100 mM CaCl₂). The hydrogel was supplemented with a proliferation medium for 5 days followed by supplementation of differentiation medium for additional 5 days.

FIG. 3 is a schematic presentation of a system according to certain embodiments of the invention for producing cultured meat product under partial, micro- or zero-gravity conditions.

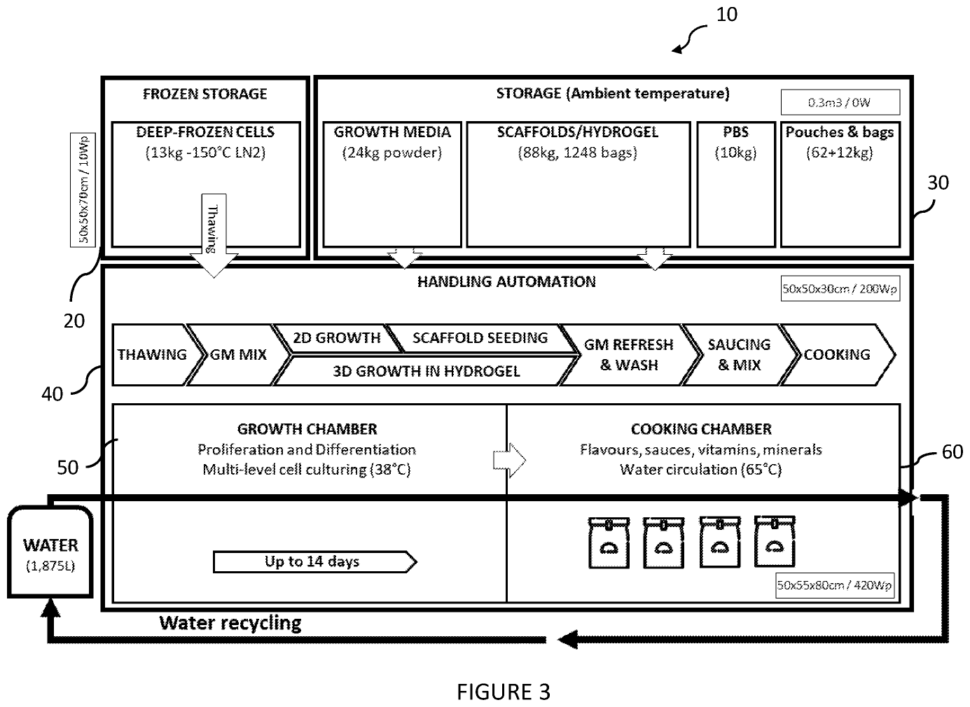

### DETAILED DESCRIPTION OF THE INVENTION

The present invention answers the need for supplementing humans spending prolonged time durations is space with proteinaceous, nutritional food. The present invention provides system and processes for the proliferation, differentiation, and maturation of non-human-animal cells, forming cultured meat products, under variable gravity conditions. The cultured meat products produced in space can be easily and directly supplemented to humans spending time in manned mission within spacecrafts, in space-stations, or in manned settlements to be established outside Erath, particularly on Moon and/or Mars. Tools for studying cell behaviors under non-earth gravity conditions for assisting in optimizing cell growth under these conditions are also provided. The variable gravity conditions include earth gravity, partial gravity, and micro- or -zero gravity conditions.

### Definitions

As used herein, the term “and/or” is intended to include any and all combinations of one or more of the associated listed items, as well as the lack of combinations when interpreted in the alternative (“or”).

As used herein, the term “consists essentially of” (and grammatical variants thereof), as applied to the compositions and processes/methods of the present disclosure, means that the compositions/processes/methods may contain additional components so long as the additional components not do materially alter the composition/process/method.

As used herein, the terms “comprise,” “comprises,” “comprising,” “contain”, “include,” “includes” and “including” specify the presence of stated features, steps, operations, elements and/or components, but do not preclude the presence or addition of one or more other features, steps, operations, elements, components and/or groups thereof.

As used herein, the term “about,” when used in reference to a measurable value such as an amount of mass, concentration, time, temperature, and the like, is meant to encompass variations of ±0.1%, 0.25%, 0.5%, 0.75%, 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19% or 20% of the specified amount. Unless otherwise indicated, all numerical values in the specification are to be understood as being modified by the term “about”. The term “approximately” is synonymous with the term “about”.

As used herein, the singular forms “a”, “an”, and “the” include plural forms unless the context clearly dictates otherwise.

As used herein, the term “plurality” refers to two or more.

As used herein, the terms “gravity” and “earth gravity” refer to gravity acceleration of 9.81 ms⁻² as experience on earth.

Accordingly, the term “partial gravity” refers to any gravity level between the theoretical zero up to Earth's unit gravity of 9.81 ms⁻². According to certain exemplary embodiments, the term “partial gravity” as used herein refers to the gravity found on Moon, which is about ⅙ of Erath gravity, or 1.62 m/s². According to yet additional exemplary embodiments, the term “partial gravity” as used herein refers to the gravity found on Mars, which is about 1/2.6 of Erath gravity, or 3.72076 m/s².

As used herein, the term “microgravity” refers to condition of which objects are nearly weightless.

As used herein, the term zero-gravity refers to the state or condition of lacking apparent gravitational acceleration.

The terms “stem cell” and “pluripotent stem cell (PSC)”, in singular or plural, are used herein interchangeably and refer to cell that is in an undifferentiated or partially differentiated state and has the capacity for self-renewal and/or to generate differentiated progeny. Self-renewal is defined as the capability of a stem cell to proliferate and give rise to more such stem cells, while maintaining its developmental potential.

As used herein, the term “differentiation conditions” refers to conditions enabling pluripotent stem cells, particularly non-human-animal pluripotent stem cells (NHAPSCs), to take a more committed (“differentiated”) position within a linage, particularly into a mesodermal lineage according to the teachings of the invention (“mesoderm committed cells”).

As used herein, the term “maturation conditions” refers to conditions enabling further differentiation of mesoderm committed cells, particularly the non-human-animal mesoderm committed cells of the present invention (MCNHACs) into a desired lineage, particularly differentiation to cells of a type selected from the group consisting of muscle cells, adipocytes, fibroblasts, endothelial cells, collagen producing cells and any combination thereof.

As used herein, the term “serum-free” with regard to a medium refers to a medium with no animal sera.

As used herein, the term “animal-derived component-free” with regard to a medium refers to a medium not containing any component of animal origin, particularly to a medium not containing mammal-derived components.

As used herein, the term “edible” refers to a material that is safe to be orally consumed by an animal, particularly by mammals, more particularly by human.

According to certain aspects, the present invention provides a process for producing non-human-animal mesoderm-committed cells under variable gravity conditions, the process comprising the steps of:
a. seeding a plurality of non-human-animal stem cells (NHASCs) in a suspension comprising cell-culture medium comprising at least one type of polysaccharide;
b. inducing conditions enabling the transition of the suspension to a solid or semisolid state, thereby forming a solid or semisolid hydrogel comprising the NHASCs;
c. maintaining the solid or semisolid hydrogel comprising the NHASCs under proliferation conditions for a duration enabling proliferation of said NHASCs; and
d. replacing the proliferation conditions to differentiation conditions and maintaining the differentiation conditions for a duration enabling the differentiation of said NHASCs to mesoderm committed non-human animal cells (MCNHACs);
thereby producing a solid or semisolid member comprising a plurality of MCNHACs.

According to certain embodiments, the duration enabling the proliferation of the NHAPSCs is at least 4 days, at least 5 days, at least 6 days, at least 7 days, at least 8 days, at least 9 days, at least 10 days, at least 11 days, at least 12 days, at least 13 days, at least 14 days, at least 15 days, or more.

According to certain embodiments, the duration enabling the proliferation of the NHASCs is from about 4 days to about 15 days. According to some embodiments, the duration enabling the proliferation of the NHASCs is from about 5 days to about 14 days, from about 5 days to about 13 days, from about 5 days to about 12 days, from about 5 days to about 11 days, from about 5 days to about 10 days, from about 5 days to about 9 days, from about 5 days to about 8 days, from about 5 days to about 7 days, or from about 5 days to about 6 days.

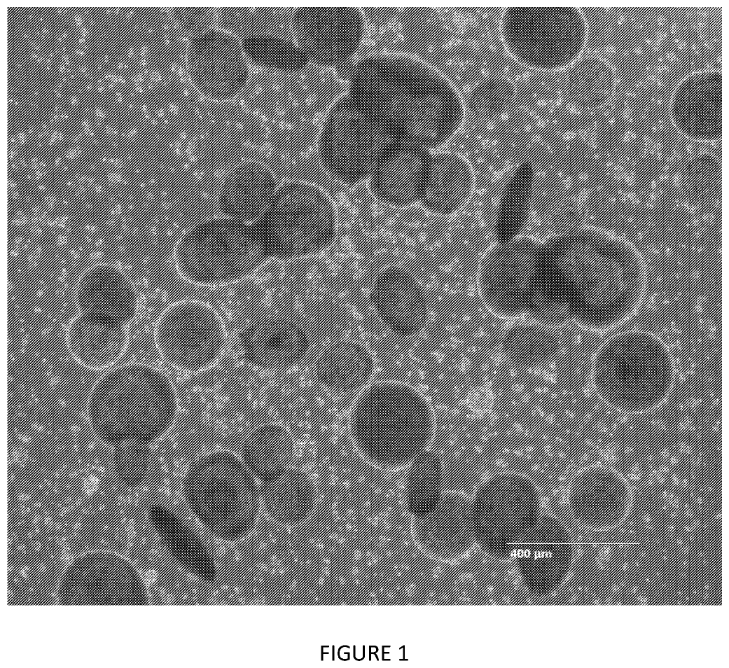

FIG. 1 shows an exemplary picture of bovine cells proliferated within a semi-solid alginate hydrogel. The picture was taken 11 days after seeding of bovine stem cells aggregates.

According to certain embodiments, the duration enabling the differentiation of the NHAPSCs to MCNHACs is at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, or more.

According to certain embodiments, the duration enabling the differentiation of the NHASCs to MCNHACs is from about 3 days to about 15 days. According to some embodiments, the duration enabling the differentiation of the NHASCs to MCNHACs is from about 4 days to about 14 days, from about 4 days to about 13 days, from about 4 days to about 12 days, from about 4 days to about 11 days, from about 4 days to about 10 days, from about 4 days to about 9 days, from about 4 days to about 8 days, from about 4 days to about 7 days, from about 4 days to about 6 days or from about 4 days to about 5 days.

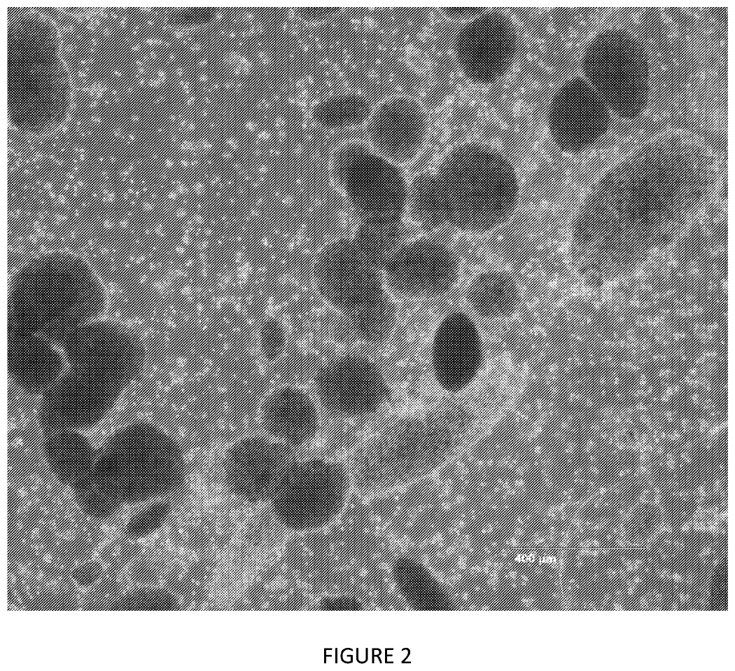

FIG. 2 shows an exemplary picture of bovine cells proliferated and differentiated within a semi-solid alginate hydrogel. The picture was taken at day 10 after seeding of bovine stem cells aggregates, wherein during the first 5 days the hydrogel was supplemented with proliferation medium and at the next 5 days the hydrogel was supplemented with differentiation medium.

The process of the present invention is designed as to enable performing its steps under variable gravity conditions. In certain exemplary embodiments, step (a) of seeding the plurality of NHASCs and/or step (b) of inducing conditions enabling the transition of the suspension to a solid or semisolid state are performed either under earth gravity conditions or under partial, micro- or zero-gravity conditions, and steps (c) and (d) are performed under partial, micro- or zero-gravity conditions.

A system to be used for performing the process of the invention may be configured to perform at least part of the process steps automatically or to perform all the process steps automatically. According to certain embodiments, at least part of steps (a)-(d) are performed within a closed, automatic system. According to certain embodiments, the entire process is performed within a closed automatic system.

According to certain embodiments, the system is configured to:
automatically inject proliferation medium through the solid or semisolid hydrogel comprising the NHASCs; and/or
automatically inject differentiation medium through the solid or semisolid hydrogel comprising the NHASCs and/or the MCNHACs; and/or
maintain a pre-set temperature within the system and compartments thereof, wherein the temperature within each compartment may be the same or different; and/or
maintain pre-set gas combination within the system and compartments thereof, wherein the gas combination within each compartment may be the same or different; and/or
automatically obtain microscopic images of the solid or semisolid hydrogel and/or the cells therein

According to certain embodiments, the system is configured to inject each of the proliferation medium and the differentiation medium at pre-determined timing (continuously or at intervals), volume, and/or flow rate as is known to a skilled in the Art, based on the system design and the cell growth parameters and conditions, including, but not limited to, the hydrogel volume, the initial cells concentration, the cell proliferation rate, and any combination thereof.

According to certain embodiments, the system is configured to inject the proliferation medium and/or differentiation medium continuously. The system may be configured to continuously inject the proliferation/differentiation medium throughout the respective growth duration, or in parts thereof with interval injections preceding or following the continuous injection.

According to certain embodiments, the system is configured to inject the proliferation medium at intervals set to once in every about 1 hours, every about 2 hours, every about 3 hours, every about 4 hours, every about 5 hours, every about 6 hours, every about 7 hours, every about 8 hours, every about 9 hours, every about 10 hours, every about 11 hours, every about 12 hours, every about 13 hours, every about 14 hours, every about 15 hours, every about 16 hours, every about 17 hours, every about 18 hours, every about 19 hours, every about 20 hours, every about 21 hours, every about 22 hours, every about 23 hours, every about 24 hours. Each possibility represents a separate embodiment of the present invention. According to certain exemplary embodiments, the intervals are set to inject the proliferation medium once every 12 hours.

According to certain embodiments, the system is configured to inject the differentiation medium at intervals set to once in every about 1 hours, every about 2 hours, every about 3 hours, every about 4 hours, every about 5 hours, every about 6 hours, every about 7 hours, every about 8 hours, every about 9 hours, every about 10 hours, every about 11 hours, every about 12 hours, every about 13 hours, every about 14 hours, every about 15 hours, every about 16 hours, every about 17 hours, every about 18 hours, every about 19 hours, every about 20 hours, every about 21 hours, every about 22 hours, every about 23 hours, or every about 24 hours. Each possibility represents a separate embodiment of the present invention. According to certain exemplary embodiments, the intervals are set to inject the proliferation medium once every 24 hours. According to certain embodiments, maintaining the gas combination comprises monitoring and adjusting CO2 level within the system or compartments thereof.

It is to be explicitly understood that the process steps can be performed by a single system configured to perform all the above-described steps, by a plurality of systems each configured to perform one or more steps, or manually.

The process of the present invention can be used for scientific purposes of studying cell proliferation and differentiation under partial gravity conditions, particularly the gravity conditions on Moon, Mars, or within a spacecraft orbiting earth; or for functional production of proteinaceous food, particularly cultured meat, under such partial gravity conditions. The cells to be used in the process of the invention are non-human-animal cells, particularly bovine cells.

According to certain exemplary embodiments, the process of the present invention is used for the production of cultured meat food. According to theses embodiments, the cells seeded in step (a) are non-human-animal pluripotent stem cells. According to certain embodiments, the non-human-animal pluripotent stem cells are obtained from an animal selected from the group consisting of ungulate, poultry, aquatic animals, invertebrate and reptiles. Each possibility represents a separate embodiment of the present invention.

According to certain embodiments, the ungulate is selected from the group consisting of a bovine, an ovine, an equine, a pig, a giraffe, a camel, a deer, a hippopotamus, and a rhinoceros. Each possibility represents a separate embodiment of the present invention. According to certain exemplary embodiments the non-human-animal stem cells are bovine cells. According to further exemplary embodiments, the bovine is a cow.

According to certain embodiments, the cells are seeded under conditions enabling the formation of aggregates, organoids, spheroids, embryonic bodies and the like within the solid or semisolid member. The conditions for the formation of aggregates of non-human-animal-derived pluripotent stem cells are essentially as described in International (PCT) Application Publication No. WO 2020/230138 to the Applicant of the present invention.

According to certain embodiments, the at least one type of polysaccharide is selected from the group consisting of alginate, starch, bean, gum, gellan-gum, hyaluronic acid, cellulose, chitin, chitosan, xanthan gum, agar, agarose, pectin, dextran, carrageenan, salts thereof, modifications and/or variations thereof, and combinations thereof. Each possibility represents a separate embodiment of the present invention. The polysaccharide modification can affect the polysaccharide melting point, viscosity, cell load capacity and more.

According to certain exemplary embodiments, the at least one polysaccharide is edible such it may retain in the final cultured meat product.

According to certain exemplary embodiments, the at least one polysaccharide is selected from the group consisting of alginate and RGD-modified alginate.

According to further exemplary embodiments, the at least one polysaccharide is alginate. The present invention now shows that proliferation of the NHAPSCs within the solid- or semi-solid hydrogel is obtained with an initial concentration of alginate in the cell suspension is up to about 0.8% w/v out of the total volumes of the cell suspension. According to certain embodiments, the concentration of the at least one polysaccharide is from about 0.3 to about 0.8% w/v, from about 0.3 to about 0.7% w/v, from about 0.3 to about 0.6% w/v, from about 0.3 to about 0.5% w/v, or from about 0.35% to about 4.5% w/v based on the volume of the suspension.

According to certain exemplary embodiments, the at least one polysaccharide is alginate, present at a concentration of 0.4% w/v based on the volume of the suspension.

According to certain embodiments, the conditions enabling the transition of the suspension to a solid or semisolid state comprise exposing said suspension to at least one crosslinking mechanism. According to some embodiments, the crosslinking mechanism is selected from the group consisting of chemical crosslinking, thermal crosslinking, photopolymerization, enzymatic polymerization, and combinations thereof. Each possibility represents a separate embodiment of the present invention. According to certain exemplary embodiments, the chemical crosslinking comprises adding to the suspension at least one divalent ion selected from the group consisting of calcium (Ca⁺²), magnesium (Mg⁺²), Barium (Ba⁺²), Copper (Cu⁺²), iron (Fe⁺²), and salts thereof. Each possibility represents a separate embodiment of the present invention.

According to certain exemplary embodiments, the chemical crosslinking comprises adding to the suspension at least one trivalent ion selected from the group consisting of iron (Fe⁺³) and Aluminum (Al⁺³). Each possibility represents a separate embodiment of the present invention.

According to further exemplary embodiments, the divalent ion is calcium (Ca⁺²) or a salt thereof.

According to certain embodiments, the cell-culture medium is a serum free medium. According to some embodiments, the cell culture medium further comprises at least one supplement selected from the group consisting of a colorant, a vitamin, a flavoring agent, folate, zinc and/or a salt thereof, selenium and/or a salt thereof, Coenzyme Q10, at least one fatty acid, yeast extract, bacterial extract, and any combination thereof. Each possibility represents a separate embodiment of the present invention.

The proliferation and the differentiation media of the invention are based on a cell-culture medium as described hereinabove supplemented with appropriate factors and small molecules to enhance proliferation and/or differentiation as are known in the art.

According to certain embodiments the process further comprises step (e) of replacing the conditions to conditions enabling further differentiation of said MCNHACs to at least one of type of cells selected from the group consisting of muscle cells, adipocytes, fibroblasts, endothelial cells, collagen producing cells and any combination thereof (maturation conditions). Each possibility represents a separate embodiment of the present invention.

The differentiation of the cells to muscle cells, adipocytes, fibroblasts, endothelial cells, collagen producing cells and combinations thereof lead to the production of high proteinaceous, edible tissue.

According to certain embodiments, the cultured meat produced by the process of the present invention comprises an edible cross-linked polysaccharide and non-human-animal cells selected from the group consisting of mesenchymal-committed non-human-animal cells (MSNHACs), muscle cells, adipocytes, fibroblasts, endothelial cells, collagen producing cells and any combination thereof. Each possibility represents a separate embodiment of the present invention.

According to certain embodiments, the cultured meat produced by the process of the present invention comprises an edible cross-linked polysaccharide and MCNHACs. According to certain additional or alternative embodiments, the cultured meat portion produced by the process of the present invention comprises an edible crosslinked polysaccharide and at least one type of cells selected from the group consisting of muscle cells, adipocytes, fibroblasts, endothelial cells, collagen producing cells and any combination thereof.

The cultured meat may be further supplemented with colorants, vitamins, flavoring agents, minerals, amino acids, fatty acids and the like, as to enhance the meat-like appearance and properties of the produced food.

According to additional aspects, the present invention provides a cultured meat product comprising at least one cross-linked edible polysaccharide and at least one type of non-human animal cells, produced by the process of the present invention.

According to certain embodiments, the non-human-animal cells within the cultured meat product are selected from the group consisting of mesenchymal-committed cells, muscle cells, adipocytes, fibroblasts, endothelial cells, collagen producing cells and any combination thereof, and any combination thereof. Each possibility represents a separate embodiment of the present invention.

According to certain embodiments, the cultured meat product comprises at least one cross-linked edible polysaccharide and mesenchymal-committed non-human animal cells (MCNHACs).

According to certain embodiments, the cultured meat product comprises at least one cross-linked edible polysaccharide and non-human-animal cells selected from the group consisting of muscle cells, adipocytes, fibroblasts, endothelial cells, collagen producing cells and any combination thereof.

According to certain embodiments, the cultured meat product comprises at least one cross-linked edible polysaccharide, MCNHACs and at least one type of non-human-animal cells selected from the group consisting of muscle cells, adipocytes, fibroblasts, endothelial cells, collagen producing cells and any combination thereof.

The cross-linked edible polysaccharide is as described hereinabove.

According to certain embodiments, the cultured meat product produced by the process of the present invention further comprises at least one supplement selected from the group consisting of a colorant, a vitamin, a flavoring agent, folate, zinc and/or a salt thereof, selenium and/or a salt thereof, Coenzyme Q10, at least one fatty acid, yeast extract, bacterial extract, at least one amino acid, at least one non-animal protein and any combination thereof. Each possibility represents a separate embodiment of the present invention.

It is to be explicitly understood that the cultured meat product may be produced by a process partially or entirely performed under micro- and/or zero-gravity conditions. According to certain embodiments, the micro- and/or zero-gravity conditions are outer space conditions. According to these embodiments, the process is performed within a system. According to some embodiments, the process by which the cultured meat is produced is performed in its entirety within a closed system under outer-space conditions.

According to yet further aspects, the present invention provides a cultured meat product comprising at least one cross-linked edible polysaccharide and at least one type of non-human animal cells, wherein the cultured meat product is essentially devoid of non-animal protein. The non-human animal cells are as described hereinabove.

As used herein, “non-animal protein” refers to a protein obtained from a plant, a fungus, an alga, or from a single cell microorganism protein. Each possibility represents a separate embodiment of the present invention.

According to further aspects, the present invention provides a method for obtaining RNA and/or RNA-marker profile of cells differentiating under variable gravity conditions, comprising the steps of:
in a first system and a second system for cell and/or tissue culturing comprising at least one compartment:
a. seeding a plurality of non-human-animal stem cells (NHASCs) in a suspension comprising cell-culture growth medium comprising at least one type of polysaccharide;
b. inducing conditions enabling the transition of the suspension to a solid or semisolid state, to thereby forming a solid or semisolid hydrogel comprising the NHASCs;
c. maintaining said solid or semisolid member comprising the NHASCs under incubation conditions for a duration enabling proliferation of said NHASCs;
d. replacing the proliferation conditions to differentiation conditions and maintaining the differentiation conditions for a duration enabling the differentiation of said NHASCs to mesoderm committed non-human animal cells (MCNHACs) and optionally to cells differentiated therefrom to obtain solid or semisolid hydrogel comprising differentiated non-human animal cells; and
e. stabilizing cellular RNA in situ within said semisolid or solid under unfrozen conditions;
wherein in the first system at least part of steps (a)-(e) are performed under gravity conditions selected from the group consisting of partial, micro- or zero-gravity conditions and in the second system the entire steps (a)-(e) are performed under earth gravity conditions.

The proliferation and differentiation conditions and durations are as described hereinabove.

According to certain exemplary embodiments, stabilizing the plurality of differentiated non-human-animal cells comprised within the solid pr semisolid hydrogel is performed using RNALater (Invitrogen). The inventors of the present invention have found that, unexpectedly, the RNALater ingredients dissolved the semi-solid or solid hydrogel made of sodium alginate, such that the cells are easily extracted for further analyses.

According to certain embodiments, the method further comprises sampling RNA from the cells comprised within the solid or semisolid hydrogel of each of the first and the second systems. According to certain embodiments, the sampled RNA is subjected to a quantification analysis. According to certain embodiments, the sampled RNA is subjected to at least one of RNA sequencing and RNA markers analysis thereby obtaining RNA and/or RNA-marker profile characteristic to cells comprised in each of the first and the second systems. According to certain embodiments, the method further comprises comparing the RNA and/or RNA-marker profile obtained from the first system to the RNA and/or RNA-marker profile obtained from the second system.

According to certain exemplary embodiments, the partial, micro- or zero-gravity conditions are outer-space conditions.

According to yet further aspects, the present invention provides a system 10 for producing cultured meat product under partial, micro- or zero-gravity conditions, the system comprising:
a. at least one storage compartment configured for storing frozen non-human-animal stem cells;
b. at least one growth compartment configured, under sterile or semi-sterile condition to (i) process NHAPSCs seeding, proliferation, and differentiation, as to obtain mesenchymal-committed non-human-animal cells (MCNHACs) and optionally cells differentiated therefrom and/or a tissue comprising same forming a meat portion; and (ii) maintain the differentiated non-human-animal cells and/or tissue comprising same and/pr cultured meat product;
c. at least one cooking compartment with means for cooking the meat portion;
d. means for transforming the non-human-animal stem cells from the at least one storage compartment to the at least one growth compartment;
e. means for transforming the meat portion from the growth compartment to the at least one cooking compartment;
f. means for supplying at least one edible liquid substance into the growth compartment; optionally,
g. means for supplying at least one edible solid substance into the growth compartment;
wherein the system is a closed system and wherein the compartments and means are suitable for operation under partial, micro- or zero-gravity conditions.

An exemplary configuration of the system is presented in FIG. 1. In some embodiments, system 10 comprises: a frozen storage unit 20; an ambient storage compartment 30; an automated handling system 40; a growth chamber 50; and a cooking chamber 60. In some embodiments (not shown), system 10 comprises a control unit. In some embodiments, the control unit comprises one or more processors and a memory. In some embodiments, the memory has stored therein instructions, that when read by the one or more processors of the control unit, cause the processors to perform various functions, such as the functions described below.

In some embodiments, frozen storage unit 20 comprises a cryo-preservation tank serving to store cell vials for long durations. In some embodiments, vials can be automatically extracted from the cryo-preservation tank by the control unit and thawed on demand. In some embodiments, ambient storage compartment 30 comprises all dry components, including growth medium powders, hydrogel powder, and optionally scaffold or scaffold components, pouches and bags, as well as at least one buffer, typically PBS buffer. In some embodiments, all items in the ambient storage compartment 30 are accessible by automated handling system 40. In some embodiments, the functions of automated handling system 40 are controlled by the control unit. In some embodiments, automated handling system 40 draws all necessary components from frozen storage unit 20 and ambient storage compartment 30. In some embodiments, automated handling system 40 performs the following process: thaws the drawn cryo-vials; dissolves the drawn growth medium powders in water to produce the different medium types; seeds the drawn cells in growth chamber 50 in a hydrogel. In some embodiment, automated handling system 40 replaces medium periodically.

In some embodiments, growth chamber 50 comprises an incubator that can accommodate growth of several cultures of solid or semisolid hydrogel comprising non-human-animal cells and cultured meat product at a given time. In some embodiments, the functions of growth chamber 50 are controlled by the control unit. In some embodiments, growth of cultured meat is staggered in the incubator in such a way as to provide ready-made steaks at various given times. In some embodiment, growth chamber 50 comprises one or more sensors which monitor growth conditions including temperature, humidity, pH and optionally CO₂. After growth of cultured meat products (steaks) is completed in growth chamber 50, cooking chamber 60 mixes sauces according to user-on-demand preference. Finally, cooking chamber 60 cooks the steaks to be made ready for consumption. In some embodiments, the functions of cooking chamber 60 are controlled by the control unit.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Proliferation and Differentiation of Bovine Pluripotent Cells in Space

The Applicant of the present invention participated in Rakia space project after the priority date of the present invention, during which the process of the present invention was examined. One system prepared as described hereinbelow was placed in the International Space Station (ISS). Additional system was prepared on earth once the space mission was completed, according to the steps and time lines performed in space, as to obtain an accurate earth-gravity control to the micro- or zero-gravity conditions in the ISS.

### ISS System

### Steps Performed Under Earth Gravity Conditions

Bovine stem cells were thawed from cryo-preserved cell bank and cultured for 2-4 cell passages. Cells were then seeded into a microfluidic device which includes 4 chambers. Each chamber was seeded with 20,000 cells. The cells were suspended in a growth medium comprising 0.4% alginate and loaded unto the microfluidic device. 100 mM CaCl₂) was flown into the device for alginate polymerization and hydrogel formation and washed out with growth medium. Seeding was performed two days before launch of the system within the ISS.

The microfluidic device was placed in a closed system of liquid storage and series of pumps including pre-programmed automatic liquid injections, temperature control, microscopic imaging and condition logging. The automatic system was loaded with two types of media required (proliferation medium and differentiation medium). The system was further loaded with RNALater Stabilization Solution (Invitrogen), a system for the in-situ preservation of RNA.

### Steps Performed Under Micro- or Zero Gravity Conditions (Space Conditions)

During the first phase of cell proliferation, proliferation medium was injected 2 times daily (every 12 hours) with pre-determined injection volume of 150 μl/chamber and flow rate of 20-50 μl/min. Until connection to the International Space Station (ISS) power socket (5-6 days after the beginning of this step), the proliferation medium was injected at a flow rate of 50 μl/min and the temperature of the entire system was maintained at about 37° C. by passive heating. Following ISS docking and power connection, the proliferation medium was injected at a flow rate of 20 μl/min, the medium temperature was maintained at 14° C. and the chamber temperature at 38.5° C. Microscopic cell images were captured periodically.

After 4-7 days of first phase, proliferation medium was replaced by differentiation medium, starting the second phase. This medium was supplemented by automatic injections once a day in pre-determined volume of 150 μl/chamber and flow rate of 20 μl/min. The second phase duration was about 5 days. Throughout the second phase, medium temperature was maintained at 13° C. and chamber temperature at 38.5° C. Microscopic cell images were captured periodically.

Following the differentiation (second) phase, cell RNA was stabilized in situ using RNALater. 175 μl/chamber of RNALater solution were injected at a flow rate of 20 μl/min in two consecutive injections. Since the return of the spaceship was postponed due to unfavorable weather conditions, disconnection of the chambers from the ISS occurred about a week after RNA stabilization. Following RNALater injection, system temperature was maintained at 4° C.

### Example 2: Comparing RNA Quantity of Non-Human Animal Cells Differentiated Under Earth Gravity Conditions and Under Micro-Gravity (Space) Conditions

Upon return to Earth, the ISS system was retrieved and cells stabilized as described in Example 1 hereinabove were extracted from the microfluidic device. Total RNA was extracted from the cells and quantified.

The entire procedure was repeated in a control experiment performed on earth by reproducing actual space-mission schedule, temperature, injection times and flow rates as described in Example 1 hereinabove.

In the closed system containing the microfluidic device the liquid inlet is shared between each pair of chambers, so both chambers in each pair experience the same injection profile and are therefore extracted and analyzed together. Due to technical problems that occurred in chambers 1+2 in the space-located system, these chambers were not included in the analysis. Total RNA extracted from the cells kept in the ISS under micro- or zero (space) gravity conditions was 3147 ng, while the quantity of RNA extracted from cells kept under earth gravity conditions was 3183 ng. These highly similar RNA yields suggest that cellular growth in microgravity and full gravity, under the experimental conditions described herein, is identical.

### Example 3: Preparation of a Cultivated Meat Portion in Space Using the System of the Invention

1) Frozen bovine stem cells are thawed on-demand and seeded in a sterile vessel comprising a polysaccharide-containing hydrogel using the automatic arm and placed in the growth chamber.

2) Automated system for cell and/or tissue culturing maintaining optimal growth conditions over 4-30 days during which the bovine stem cells proliferate and differentiate naturally to mesenchymal committed cells, optionally further differentiate to at least one cell type selected from the group consisting of muscle cells, adipocytes, fibroblasts, endothelial cells, collagen producing cells and any combination thereof, and any combination thereof to form cultured meat.

3) The cultured meat is transferred by the user to the cooking chamber, while keeping a pathogen-free environment by following specific protocols.

The process is based on the following assumptions:
an aseptic environment within the growth chamber. Since the growth phase requires minimal interaction by the spacecraft crew, no problem of maintaining aseptic conditions is expected. Regardless, the users are capable of performing industry standard microbiological testing.
Access to a water recycling system that provides approximately 98% water recovery. The waste streams are to be compatible with such a water recycler, since the waste does not contain harmful chemicals or any solids which could clog or damage the recycler.
Dimension. The total volume of the system is about 1 cubic meter, such that the system is compatible with the doorway and room size requirements of a spacecraft.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A process for producing mesoderm-committed non-human-animal cells (MCNHACs) under variable gravity conditions, comprising the steps of:
a. seeding a plurality of non-human-animal pluripotent stem cells (NHAPSCs) in a suspension comprising cell culture medium comprising at least one type of polysaccharide:
b. inducing conditions enabling the transition of the suspension to a solid or semisolid state, thereby forming a solid or semisolid hydrogel comprising the NHAPSCs:
c. maintaining the solid or semisolid hydrogel comprising the NHAPSCs under proliferation conditions for a duration enabling proliferation of said NHAPSCs; and
d. replacing the proliferation conditions to differentiation conditions and maintaining the differentiation conditions for a duration enabling the differentiation of said NHAPSCs to MCNHACs:
thereby producing a solid or semisolid hydrogel comprising a plurality of MCNHACs.

2-3. (canceled)

4. The process of claim 1, wherein at least one of steps (a) to (d) is performed within a system comprising at least one compartment.

5. (canceled)

6. The process of claim 4, wherein the system is configured to perform at least one of steps (a) to (d) automatically.

7. (canceled)

8. The process of claim 1, wherein the variable gravity conditions are selected from the group consisting of earth gravity conditions, partial gravity conditions, micro-gravity conditions, zero gravity conditions and a combination of earth gravity conditions and micro- or zero gravity conditions.

9-11. (canceled)

12. The process of claim 1, wherein the concentration of the at least one type of polysaccharide is from about 0.3 to about 0.8% w/v of the suspension.

13. (canceled)

14. The process of claim 1, wherein the conditions enabling the transition of the suspension to a solid or semisolid state comprise exposing said suspension to at least one crosslinking mechanism, and wherein the crosslinking mechanism is selected from the group consisting of chemical crosslinking, thermal crosslinking, photopolymerization, enzymatic polymerization, and combinations thereof.

15. (canceled)

16. The process of claim 14, wherein crosslinking mechanism is chemical crosslinking and wherein said chemical crosslinking comprises adding to the suspension at least one cation selected from the group consisting of calcium (Ca⁺²), Barium (Ba⁺²), Copper (Cu⁺²), magnesium (Mg⁺²), iron (Fe⁺² and/or Fe⁺³), Aluminum (Al⁺³), and salts thereof.

17-18. (canceled)

19. The process of claim 4, wherein the system is configured to automatically inject (i) proliferation medium through the solid or semisolid hydrogel comprising the NHAPSCs at a pre-determined injection interval, volume and/or flow rate and/or (ii) differentiation medium through the solid or semisolid hydrogel comprising the NHAPSCs and/or MCNHACs at a pre-determined interval, injection volume and/or flow rate.

20. (canceled)

21. The process of claim 4, wherein the system comprises at least one compartment comprising the solid or semisolid hydrogel comprising a plurality of non-human-animal cells and at least one additional compartment.

22. The process of claim 4, wherein the system is further configured to maintain a pre-set temperature in the at least one compartment of said system, wherein the pre-set temperature in the at least one compartment comprising the solid or semi solid hydrogel is in a range selected from the group consisting of from about 4° C. to about 15° C. and from about 35° C. to about 39° C.

23. (canceled)

24. The process of claim 4, wherein the system is further configured to automatically obtain microscopic images of the solid or semisolid hydrogel and/or the non-human-animal cells therein.

25. The process of claim 1, wherein said process further comprises step (e) of replacing the differentiation conditions to maturation conditions enabling further differentiation of said MCNHACs to at least one type of cells selected from the group consisting of muscle cells, adipocytes, fibroblasts, endothelial cells, collagen producing cells and any combination thereof.

26. The process of claim 1, wherein said process results in the production of a cultured meat product.

27-28. (canceled)

29. The process of claim 4, wherein said process in its entirety is performed within a closed system.

30. The process of claim 1, wherein the micro- or zero-gravity conditions are outer space conditions.

31. A cultured meat product comprising at least one cross-linked edible polysaccharide and at least one type of non-human animal cells, produced by the process of claim 1.

32-37. (canceled)

38. A method for obtaining RNA and/or RNA-marker profile of cells differentiating under variable gravity condition, comprising the steps of:
in a first system and a second system for cell and/or tissue culturing comprising at least one compartment:
e. seeding a plurality of non-human-animal pluripotent stem cells (NHAPSCs) in a suspension comprising cell-culture growth medium comprising at least one type of polysaccharide;
f. inducing conditions enabling the transition of the suspension to a solid or semisolid state, thereby forming a solid or semisolid hydrogel comprising the NHAPSCs:
g. maintaining said solid or semisolid hydrogel comprising the NHASCs under proliferation conditions for a duration enabling proliferation of said NHAPSCs;
h. replacing the proliferation conditions to differentiation conditions and maintaining the differentiation conditions for a duration enabling the differentiation of said NHAPSCs to mesoderm committed non-human animal cells (MCNHACs) and optionally to cells differentiated therefrom to obtain solid or semisolid hydrogel comprising differentiated non-human animal cells; and
i. stabilizing cellular RNA in situ within said solid or semisolid hydrogel under unfrozen conditions:
wherein in the first system at least part of steps (a) to (e) are performed under gravity conditions selected from the group consisting of partial, micro- or zero-gravity conditions and in the second system the entire steps (a) to (e) are performed under earth gravity conditions.

39-40. (canceled)

41. The method of claim 38, wherein said method further comprises sampling the cellular RNA of each of the first and the second systems, wherein the sampled RNA is subjected to at least one of RNA quantification, RNA sequencing, RNA marker analysis and any combination thereof.

42. (canceled)

43. The method of claim 41, wherein said method further comprises comparing the RNA quantity, the RNA sequence and/or the RNA-marker profile obtained from the first system to the RNA quantity, the RNA sequence and/or the RNA-marker profile obtained from the second system.

44-45. (canceled)

46. A system for producing cultured meat product under partial, micro- or zero-gravity conditions, the system comprising:
j. at least one storage compartment configured to store frozen non-human-animal pluripotent stem cells (NHAPSCs);
k. at least one growth compartment configured, under sterile or semi-sterile condition to (i) process NHAPSCs seeding, proliferation and differentiation as to obtain differentiated non-human-animal cell and/or a tissue comprising same forming a meat portion; and (ii) maintain the non-human-animal cells and/or tissue comprising same and/or meat portion:
l. at least one cooking compartment with means for cooking the meat portion:
m. means for transforming the NHAPSCs from the at least one storage compartment to the at least one growth compartment:
n. means for transforming the meat portion from the at least one growth compartment to the at least one cooking compartment;
o. means for supplying at least one edible liquid substance into the at least one growth compartment; optionally
p. means for supplying at least one edible solid substance into the at least one growth compartment;
wherein the system is a closed system and wherein the compartments and means are suitable for operation under partial, micro- or zero-gravity conditions.

47-50. (canceled)
